(19) United States
(12) Patent Application Publication
(10) Pub. No.: US 2024/0167991 A1
(43) Pub. Date: May 23, 2024

(54) CARBON SEQUESTRATION EVALUATION

(71) Applicant: Saudi Arabian Oil Company, Dhahran (SA)

(72) Inventors: Mohammed Sayed, Katy, TX (US); Katherine Leigh Hull, Houston, TX (US); Younane N. Abousleiman, Norman, OK (US); Nam Mai, Houston, TX (US)

(21) Appl. No.: 17/992,466

(22) Filed: Nov. 22, 2022

(51) Int. Cl. G01N 33/00; G01N 33/24

(52) U.S. Cl. CPC G01N 33/004; G01N 33/24

(57) ABSTRACT

An apparatus for determining a carbon sequestration characteristic of a geological material includes a sample holder within an inner chamber and a pressure pump configured to apply a confining pressure to the inner chamber, a first pressure gauge and a second pressure gauge configured to measure a fluid pressure at the fluid inlet and a fluid pressure at the fluid outlet, respectively, and a carbon dioxide pump configured to inject carbon dioxide into the inner chamber via the fluid inlet. The apparatus further includes a separator for separating a volume of carbon dioxide from fluid flowing from the fluid outlet, and a return line configured to return at least a portion of the volume of carbon dioxide separated by the separator to the fluid inlet.

### TECHNICAL FIELD

This disclosure relates to subterranean carbon sequestration.

### BACKGROUND

Anthropogenic carbon dioxide emissions continue to rise steadily. Potential global climate change associated with greenhouse gas (GHG) emissions can only be mitigated by reducing atmospheric carbon levels through emissions cuts or carbon capture utilization and storage (CCUS) technologies.

The aggressive goal to reduce atmospheric carbon dioxide accumulation will be very challenging to meet without significant technological developments for CCUS. Technologies for capturing and storing carbon dioxide, including those that can be retrofitted to existing structures, will be critical to achieving the global climate change mitigation goals.

Underground geological sequestration may be of particular interest for CCUS due to the potential large storage capacity of geological formations and the permanence of such sequestration. In geological sequestration, carbon in the form of carbon dioxide is injected to suitable depths within the Earth wherein it can be permanently fixated. Geological materials that have shown proven potentials for carbon storage include depleted oil and gas reservoirs, saline aquifers, and unmined coal beds.

Understanding carbon sequestration characteristics of a geological material, such as the carbon dioxide storage capacity of the material or the effect of treatment chemicals on sequestration parameters, can aid in developing more cost-effective and successful sequestration technologies.

### SUMMARY

This disclosure describes methods and apparatus for analyzing a carbon sequestration characteristic of a geological material, such as the carbon dioxide storage capacity of the material or the effect of treatment chemicals on sequestration parameters.

Certain aspects of the subject matter herein can be implemented as an apparatus for determining a carbon sequestration characteristic of a geological material. The apparatus includes a sample holder including a fluid inlet, an inner chamber, and a fluid outlet. The sample holder is configured to hold a sample representative of the geological material within the inner chamber and to permit a flow of fluid from the fluid inlet through the sample and thence out the fluid outlet. The apparatus further includes a pressure pump configured to apply a confining pressure to the inner chamber, a first pressure gauge and a second pressure gauge configured to measure a fluid pressure at the fluid inlet and a fluid pressure at the fluid outlet, respectively, and a carbon dioxide pump configured to inject carbon dioxide into the inner chamber via the fluid inlet. The apparatus further includes a separator for separating a volume of carbon dioxide from fluid flowing from the fluid outlet, and a return line configured to return at least a portion of the volume of carbon dioxide separated by the separator to the fluid inlet.

An aspect combinable with any of the other aspects can include the following features. The apparatus can further include comprising a gas flow meter to measure a flow rate of the gas flowing from the fluid outlet to the separator;

An aspect combinable with any of the other aspects can include the following features. The apparatus can further include a treatment injection pump configured to inject a treatment chemical into the fluid inlet.

An aspect combinable with any of the other aspects can include the following features. The treatment chemical can include an oxidizer.

An aspect combinable with any of the other aspects can include the following features. The oxidizer can be water-soluble.

An aspect combinable with any of the other aspects can include the following features. Carbon dioxide injected by the carbon dioxide pump can include a mixture including (a) carbon dioxide returned by the return line and (b) carbon dioxide from a carbon dioxide tank.

An aspect combinable with any of the other aspects can include the following features. The carbon dioxide from the carbon dioxide tank may not have been in contact with the sample before the injecting.

An aspect combinable with any of the other aspects can include the following features. The apparatus can further include a gas chromatograph-mass spectrometer configured to determine a composition of a fluid mixture flowing from the fluid outlet.

Certain aspects of the subject matter herein can be implemented a method for evaluating a carbon sequestration characteristic of a geological material. The method includes applying a confining pressure to an inner chamber of a sample holder, wherein a sample representative of the geological material is disposed in the inner chamber. The method further includes injecting carbon dioxide into the inner chamber via a fluid inlet of the sample holder, thereby flowing the carbon dioxide at least partially through the sample, and determining a carbon dioxide sequestration characteristic of the sample. The determining of the characteristic can be based, at least in part, on at least one of (a) a change in measured inner chamber pressure or (b) a change over time of a pressure differential across the sample. The method further includes flowing, out of the inner chamber via a fluid outlet of the sample holder, a fluid at least partially comprising the carbon dioxide flowed through the sample, separating a volume of carbon dioxide from the fluid flowing from the fluid outlet, re-injecting at least a portion of the volume of carbon dioxide into the inner chamber.

An aspect combinable with any of the other aspects can include the following features. The carbon dioxide sequestration characteristic can include a volumetric carbon dioxide storage capacity of the sample and the volumetric storage capacity can be based at least in part on a measured difference between measured inner chamber pressure before and after injection of the carbon dioxide.

An aspect combinable with any of the other aspects can include the following features. The geological formation of which the sample is representative can include a saline-saturated formation, and the carbon dioxide sequestration characteristic can include an increase in carbonate mineralization in the sample resulting from treatment of the sample by a treatment chemical. The method can further injecting a saline aqueous fluid into the inner chamber, injecting the treatment chemical into the inner chamber, measuring, over time, a pressure differential across the sample, and determining, based at least in part on variations over time of the pressure differential, the increase in carbonate mineralization.

An aspect combinable with any of the other aspects can include the following features. The geological formation of which the sample is representative can include an organic-rich formation. The carbon dioxide sequestration characteristic can include an increase carbon dioxide absorption resulting from treatment of the sample by an oxidizer, and the injecting carbon dioxide into the inner chamber can include a first injection of carbon dioxide. The method can further include determining a pre-treatment volumetric storage capacity based at least in part on a measured difference between measured inner chamber pressure before and after the first injection of the carbon dioxide, flowing, from the inner chamber, at least a portion of the carbon dioxide from the first injection, injecting an aliquot of the oxidizer into the inner chamber, injecting carbon dioxide into the inner chamber as a second injection of carbon dioxide, determining a post-treatment volumetric storage capacity based at least in part on a measured difference between measured inner chamber pressure before and after the second injection of the carbon dioxide, and determining, based at least in part on a difference between the pre-treatment storage capacity and the post-treatment storage capacity, the increase in carbon dioxide absorption.

An aspect combinable with any of the other aspects can include the following features. The method can further include increasing the organic content of the sample by injecting a hydrocarbon fluid into the inner chamber prior to the first injection of carbon dioxide.

An aspect combinable with any of the other aspects can include the following features. The oxidizer can be water-soluble.

An aspect combinable with any of the other aspects can include the following features. The method can further include measuring, with a flow meter, a flow rate of the gas flowing through the sample chamber.

An aspect combinable with any of the other aspects can include the following features. Injecting carbon dioxide into the inner chamber comprises the reinjecting of the portion of the carbon dioxide separated by the separator.

An aspect combinable with any of the other aspects can include the following features. The portion of the carbon dioxide separated by the separator is injected as a mixture with carbon dioxide from a carbon dioxide tank.

An aspect combinable with any of the other aspects can include the following features. The carbon dioxide from the carbon dioxide tank may not have been in contact with the sample before the injecting.

An aspect combinable with any of the other aspects can include the following features. The method can further include determining, with a gas chromatograph-mass spectrometer fluidically connected to the inner chamber, a composition of a fluid mixture flowing from the fluid outlet.

An aspect combinable with any of the other aspects can include the following features. The re-injecting of the at least a portion of the volume of carbon dioxide into the inner chamber can be via a return line fluidically connected to the fluid outlet.

An aspect combinable with any of the other aspects can include the following features. The return line can be configured to flow the at least a portion of the volume of carbon dioxide to a pump configured to inject the carbon dioxide into the inner chamber via the fluid inlet.

### DESCRIPTION OF DRAWINGS

FIG. 1 is a schematic illustration of an apparatus for determination of a carbon dioxide sequestration characteristic of a geological material, in accordance with an embodiment of the present disclosure.

FIG. 2 is a process flowchart of an example of a method 200 for determining a carbon sequestration characteristic of a geological sample in accordance with an embodiment of the present disclosure.

FIG. 3 is a process flowchart of an example of a method 200 for determining a carbon sequestration characteristic of a geological sample in accordance with an embodiment of the present disclosure.

FIG. 4 is a process flowchart of an example of a method 200 for determining a carbon sequestration characteristic of a geological sample in accordance with an embodiment of the present disclosure.

FIG. 5 is a process flowchart of an example of a method 200 for determining a carbon sequestration characteristic of a geological sample in accordance with an embodiment of the present disclosure

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

In accordance with embodiments of the present disclosure, and apparatus and method are disclosed for injecting carbon dioxide and other chemicals into a sample of a geological material and measuring the extent and other characteristics of such injection on the sample. For example, a carbon sequestration characteristic of a sample of geological material, such as the carbon dioxide storage capacity of the material or the effect of treatment chemicals on sequestration parameters, can be determined and/or measured. In accordance with embodiments of the present disclosure, an apparatus and method for determining the effectiveness of treatment chemicals in organic-rich or brine-saturated material are disclosed.

In some embodiments of the present disclosure, carbon dioxide exiting the sample after injection can be captured and re-injected into the sample. Such recycling of the carbon dioxide gas into the sample can enable the study the effects of continuous cycling and change in the gas composition in the sample and the impact of slow reactions that take place in a geological reservoir. Such recycling and re-injection can be further utilized to determine and measure the effect of slow chemical reactions as a function of time and hence the impact of continuous injection of carbon dioxide on rock dissolution, scale precipitation and composition changes, over long time periods or time scales within a reservoir. Such determination can be particularly useful when considering large reservoir volumes, which can be considered infinite with respect to the injection rate and time it takes for a fluid to travel from an injector to a producer. In the case of enhanced oil recovery and other applications such as carbon dioxide sequestration and mineralization, the injection rates are not high and hence the residence time of the carbon dioxide in a reservoir can be great. To simulate this in a core flood, which represents a very limited volume to represent the whole reservoir, researchers used to inject fluid at very low injection rates to increase the residence time in the reservoir and hence allow the reaction between the injected fluids and the matrix to happened and to be evaluated. A further advantage of recycling and re-injecting the carbon dioxide is that the same carbon dioxide can be injected into a small core volume repeatedly so as to aid in the study the continuous change in the carbon dioxide and water properties, especially the change in the type and concentration of cations resulting from the possible dissolution of rock in a carbon dioxide-brine mixture or precipitation of minerals, salts or any other organic matter from the reservoir fluids, and their impact on the reservoir rock properties and the pore network characterization. Other long-term measurements and determinations can include the viscosity and interfacial tension reduction of the crude as a result of carbon dioxide dissolution and miscibility in crude oil. After carbon dioxide is dissolved in crude oil, the viscosity of crude oil can be significantly reduced. Carbon dioxide dissolution in water can increase the viscosity of water, so as to improve the oil-water mobility ratio. Recycling carbon dioxide in a core flood experiment help in investigating core-scale supercritical carbon dioxide-brine-rock interactions under geological reservoir conditions during the injection of carbon dioxide and an experimental scheme for investigating carbon dioxide storage in deep saline aquifers. Such capture and recycling the carbon dioxide exiting the sample chamber can also function to reduce sample consumption and reduce or eliminate the release of carbon dioxide into the atmosphere from operation of the apparatus.

Furthermore, because laboratory apparatus and methods for conducting such determinations necessarily use volumes of carbon dioxide, the potential exists for substantial emissions of this carbon dioxide into the atmosphere. In accordance with some embodiments of the referenced disclosure, by recapturing and reutilizing carbon dioxide utilized in the determination process, overall carbon emissions from such testing can be minimized.

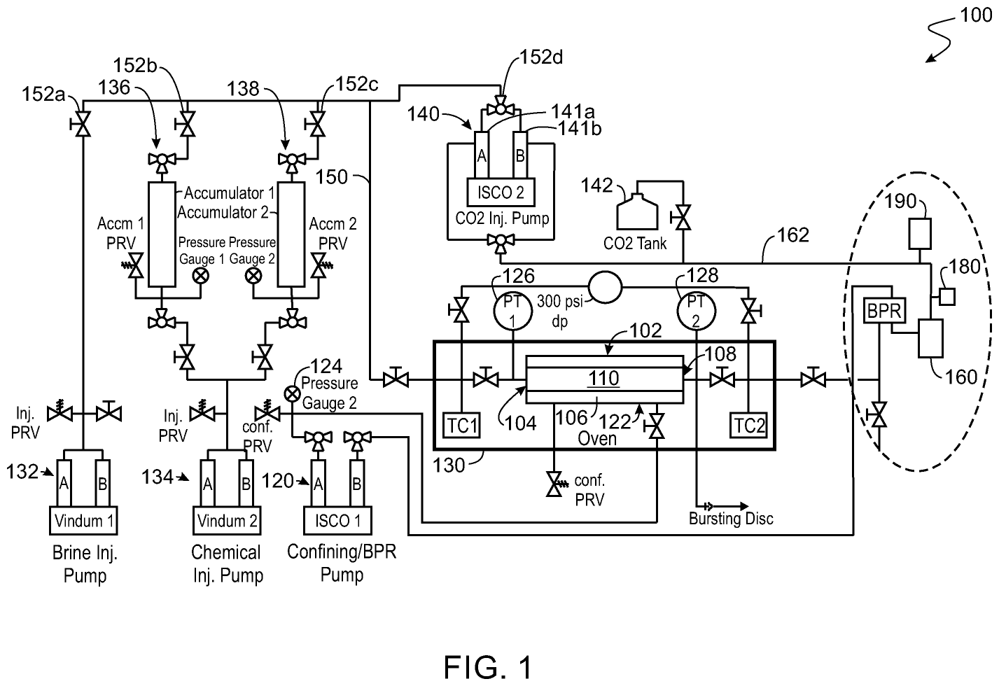

FIG. 1 is a schematic illustration of an apparatus for determination of a carbon dioxide sequestration characteristic of a geological material, in accordance with an embodiment of the present disclosure. Referring to FIG. 1, apparatus 100 includes a sample holder 102, a fluid inlet 104, an inner chamber 106, and a fluid outlet 108. Sample holder 102 is configured to hold a sample 110 within inner chamber 106. Sample 110 can be, for example, a core sample from a geological formation or a sample manufactured from natural or human-made materials to simulate (in terms of composition, porosity, permeability, fluid content, and/or other parameters) a natural geological material. Sample holder 102 is configured to permit a flow of fluid from the fluid inlet 104 through the sample 110 and thence out the fluid outlet 108.

In the illustrated embodiment, apparatus 100 further includes a confining pressure pump 120 configured to apply a confining pressure to the inner chamber 106 via a confining pressure inlet 122. Confining pressure gauge 124 is configured to measure the confining pressure within the inner chamber 106, thus allowing the operator to control the pressure as may be desired or required to simulate the natural subsurface pressure of the geological material being analyzed or considered and determine the pore pressure within the sample. Inlet pressure gauge 126 and outlet pressure gauge 128 are configured to measure a fluid pressure at the fluid inlet 104 and a fluid pressure at the fluid outlet 108, respectively, thus allowing the operator to determine the differential pressure across the sample within chamber 106. In the illustrated embodiment, apparatus 100 further includes an oven 130 within which sample holder 102 is positioned, thus allowing the operator to control the pressure as may be desired to simulate the natural subsurface temperature of the geological material being analyzed or considered.

In the illustrated embodiment, apparatus 100 further can include one or more fluid injection pumps, which in the illustrated embodiment include fluid injection pump 132, treatment injection pump 134, and carbon dioxide injection pump 140. Fluid injection pump 134 can inject fluids (gases or liquids) into first accumulator 136 and the same or different gases or liquids into second accumulator 138, which can then in turn inject the chemicals separately or together into the chamber so as to fill or partially fill the pores of the sample with an amount and kind of fluids present in the geological material under subsurface conditions and/or with suitable treatment chemicals. Such fluids can include carbon dioxide, brine, water, oxidizers, mineralization accelerants, or other chemicals (liquids or gases) into the chamber for studies of the effects of such treatments on the sample in question. Carbon dioxide injection pump 140 can inject a selected volume of carbon dioxide (from carbon dioxide tank 142) into the chamber so as to flow carbon dioxide through the sample, which can simulate the carbon dioxide injection process of an actual subsurface sequestration operation. In some embodiments, carbon dioxide tank 142 and carbon dioxide injection pump 140 are configured so as to store and inject carbon dioxide in its supercritical form. Valves (for example, 152a, 152b, 152c, and 152d) can selectively control fluid flow from these pumps into feed line 150, which provides a pathway for the fluids to flow into inlet 104. Apparatus 100 can include various pressure relief valves and other suitable valves on piping and other fluid conveyances of apparatus 100 to selectively control fluid flow through various fluid flow lines and other components of apparatus 100.

In the illustrated embodiment, apparatus 100 includes a separator 160 for separating some or all of the carbon dioxide from the fluid flowing from the fluid outlet 108. Flow meter 180 can measure the volume of carbon dioxide exiting the sample chamber 110 as a function of time. Such measurements can be used to calculate, for example, gas storativity of the sample before and after treatment with treatment chemicals.

In the illustrated embodiment, apparatus 100 includes gas chromatograh—mass spectrometer (GC-MS) 190 to determine the composition of the gas and gas mixtures exiting the core sample. Such composition information can be used to, for example, determine the nature and extent of chemical reactions occurring within the core sample and/or the effect of treatment chemicals on the sample and/or on such reactions.

In the illustrated embodiment, apparatus 100 includes a return line 162 which can be configured to return at least a portion of the volume of carbon dioxide exiting the sample back to the apparatus to be re-injected back into the sample in sample chamber 108. Such recycling and re-injection of the carbon dioxide gas into the sample can simulate continuous and slow cycling and re-cycling of carbon dioxide flowing within a subterranean reservoir, thus enabling, for example, the study of relatively slow and continuous reactions that can take place over time in a geological reservoir, as described above. Such capture and recycling the carbon dioxide exiting the sample chamber can also function to reduce sample consumption and reduce or eliminate the release of carbon dioxide into the atmosphere from operation of the apparatus. In the illustrated embodiments, carbon dioxide pump 140 can include separate cylinders—cylinder 141a and cylinder 141b—configured to injecting separate volumes of carbon dioxide into sample chamber 106. For example, in some embodiments, cylinder 141a can receive recycled carbon dioxide from return line 162 and cylinder 141b can receive “fresh” carbon dioxide from tank 142, such that a mixture of recycled carbon dioxide separated from fluid outlet 108 and “fresh” carbon dioxide from tank 142 can be injected together or separately into inner chamber 106.

It will be understood that some embodiments of the present disclosure may omit some of the components or features that are present in the embodiment shown in FIG. 1. For example, in some embodiments, the apparatus can omit one or more of separator 160, return line 162, flow meter 180, and/or GCMS 190.

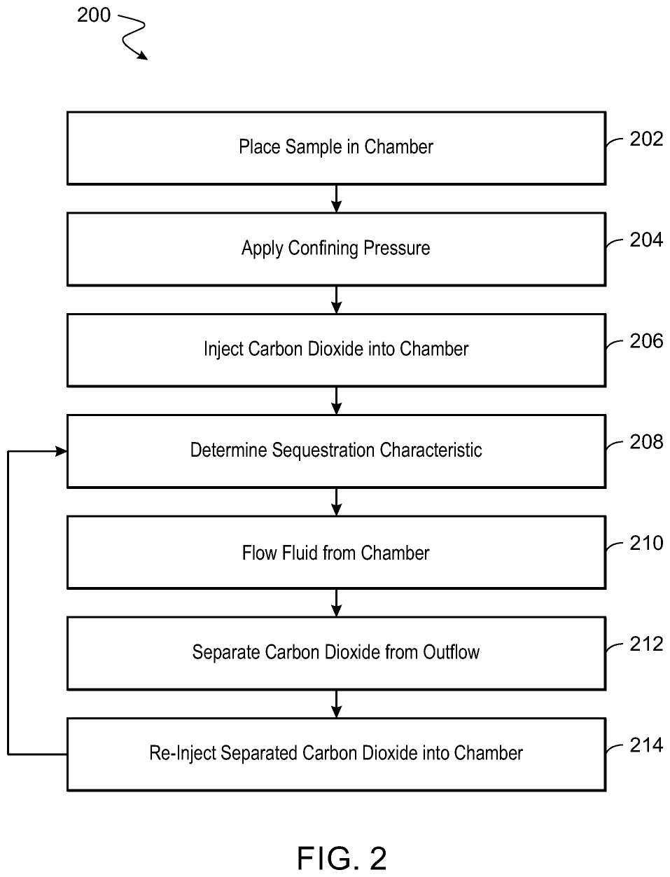

FIG. 2 is a process flowchart of an example of a method 200 for determining a carbon sequestration characteristic of a geological sample in accordance with an embodiment of the present disclosure. The characteristic can include, for example, an extent of carbon sequestration over time for such geological material and/or the effectiveness of chemical treatment on carbon sequestration in the geological material. The sample can be, for example, sample 110 (which as described above can be, for example, a core sample from a geological formation or a sample manufactured from natural or human-made materials to simulate (in terms of composition, porosity, permeability, fluid content, and/or other parameters) a natural geological material. Method 200 is described herein in reference to apparatus 100 and its components as described above; however, it will be understood that method 200 may be performed with another suitable apparatus or other suitable components.

The method begins at step 202 wherein the sample is placed in inner chamber 106. At step 204, a confining pressure is applied to inner chamber 106 to simulate the geological context. At step 206, carbon dioxide (such as supercritical carbon dioxide) is injected into chamber 106 under the desired pressure and temperature.

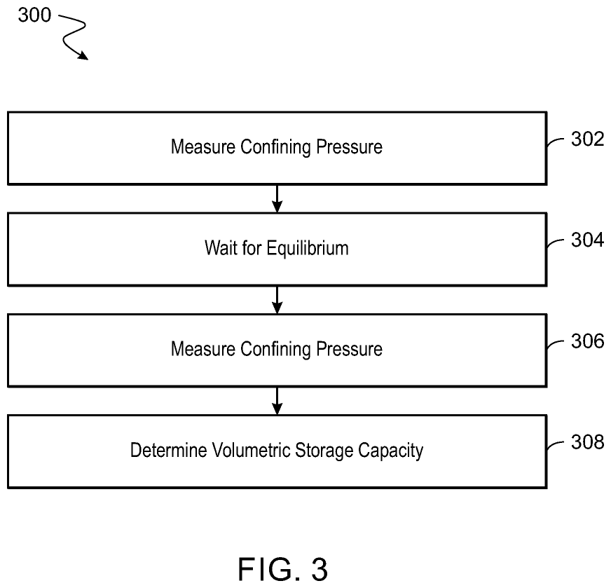

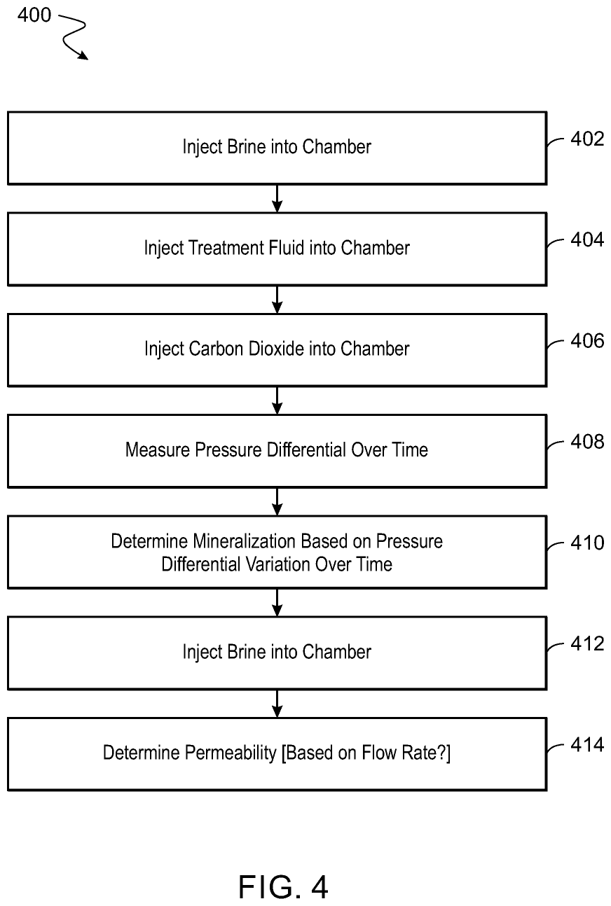

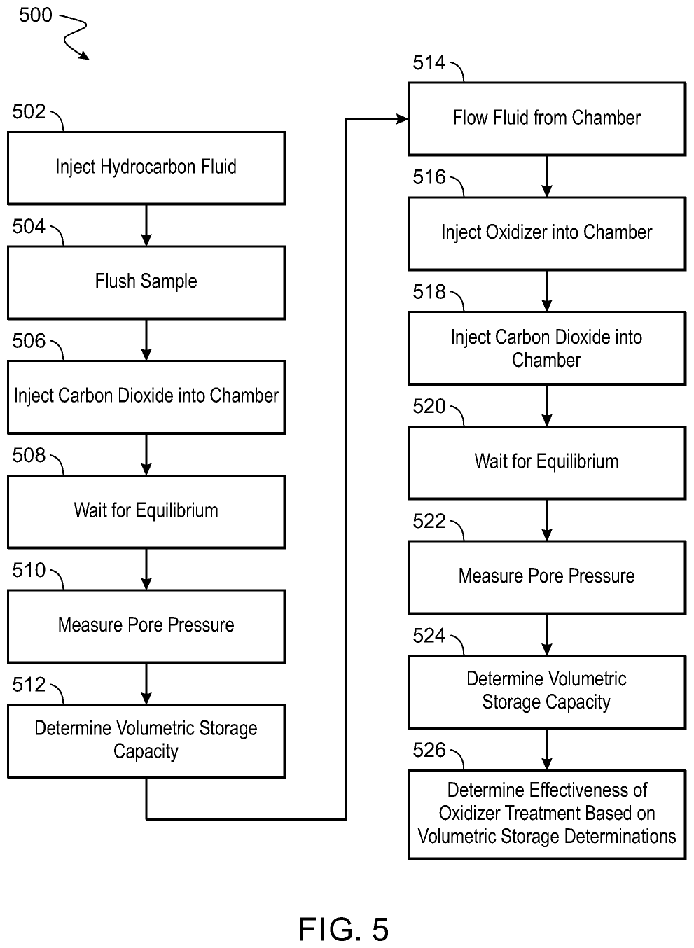

At step 208, a sequestration characteristic of sample 110 is determined. For example, in some embodiments, the sequestration characteristic can be a volumetric capacity of the sample to contain carbon dioxide, in light of the porosity, permeability, and other characteristics of the sample. A method for such a volumetric capacity determination in accordance with an embodiment of the present disclosure is described in greater detail in reference to FIG. 3. In some embodiments, the sequestration characteristic can be the effect of a treatment chemical on carbonate mineralization reactions within a brine-saturated sample and corresponding changes in permeability. A method for such a treatment chemical evaluation in accordance with an embodiment of the present disclosure is described in greater detail in reference to FIG. 4. In some embodiments, the sequestration characteristic can be the effect of an oxidizer on sequestration of carbon in organic-rich geological material. A method for such oxidizer evaluation in accordance with an embodiment of the present disclosure is described in greater detail in reference to FIG. 5. In some embodiments, and as described in further detail below, one or more of the carbon dioxide sequestration characteristics (or other carbon dioxide sequestration characteristics) can be determined, at least in part, on a change in measured inner chamber pressure, a change over time of a pressure differential across the sample, and/or other parameters or measurements.

Returning to FIG. 2, at step 210, after the sequestration characteristic is determined, fluid in the chamber that has been flowed through sample 110 is flowed out of inner chamber 106 via fluid outlet 108, and, at step 212, all or a portion of the carbon dioxide in the fluid is separated from the fluid by separator 160. At step 214, at least a portion of the separated carbon dioxide is re-injected into inner chamber 106. Such re-injection can provide all or a portion of the carbon dioxide subsequently injected into chamber 106 for the same or a different sequestration characteristic determination sequence or sequences. In some embodiments, the injection at step 214 can include a mixture of recycled carbon dioxide separated from fluid outlet 108 and “fresh” carbon dioxide from tank 142 can be injected into inner chamber 106. In some embodiments, the injection at step 214 can include only recycled carbon dioxide. After step 214, the method can then return to step 208 for further and/or continuous determination of the sequestration characteristic using the recycled carbon dioxide (or the mixture of the recycled carbon dioxide with fresh carbon dioxide). As described above, the continued sequestration determination using the recycled carbon dioxide can enable analysis of slow surface reactions, continuous injections, or other long term or long time-scale reactions and subsurface. If analysis and determination is complete, the method can end at step 214 without returning to step 208.

FIG. 3 is a process flowchart of an example of a method 300 for determining a carbon sequestration characteristic of a geological material; specifically, a volumetric capacity such material to contain carbon dioxide. In some embodiments, method 300 provides the specific sub-steps corresponding to step 208 of FIG. 2, with respect to such type of determination.

Method 300 can be applied to a sample that has been placed into a sample holder chamber, a confining pressure applied, and into which carbon dioxide has been injected (for example, in accordance with steps 202, 204, and 206 (and/or recycling and re-injection step 214) of method 200 of FIG. 2). In some embodiments, heat can also be applied to the sample at a suitable time to simulate subsurface temperatures. After the sample is so placed, pressurized, and heated, then beginning at step 302, the initial confining pressure after the carbon dioxide injection is measured. At step 304, the operator waits a suitable amount of time to allow for the injected carbon dioxide to saturate the sample, at the given pressure, temperature, and sample characteristics. At step 304, the confining pressure is again measured after the time allotted to equalization has passed. At step 308, the volumetric storage capacity of the sample is determined based on the difference between these confining pressure measurements, for example, using the real gas law subjected to reservoir conditions.

FIG. 4 is a process flowchart of an example of a method 400 for determining a carbon sequestration characteristic of a geological material; more specifically, the effectiveness of treating a brine-saturated geological material with a treatment fluid on the carbon storativity/absorption capacity of the material. In some embodiments, method 400 provides the specific sub-steps corresponding to step 208 of FIG. 2, with respect to such type of determination.

Method 400 can be applied to a sample that has been placed into a sample holder chamber and a confining pressure applied (for example, in accordance with steps 202 and 204 (and/or recycling and reinjection step 214) of method 200 of FIG. 2). In some embodiments, heat can also be applied to the sample at a suitable time to simulate subsurface temperatures. After the sample has been so placed, pressurized, and heated (if applicable) then, at step 402, brine or another suitable saline aqueous solution is injected into the inner chamber of the sample holder, such that the sample becomes saturated. At step 404, a predetermined volume of the treatment fluid is injected to mix with the brine within the pores of the sample. In some embodiments, the treatment fluid can comprise a mineralization accelerant such as commercially available amino acids and/or their sodium or potassium salts (for example, monoethanolamine, functionalized monoethanolamines, primary or secondary amine containing alkyl, aromatic, or combination of both, or sodium glycinate). At step 406, carbon dioxide (for example, supercritical carbon dioxide) is injected at an injection rate that is low enough to allow sufficient time for the carbon dioxide to react with the divalent and trivalent cations in the brine, thereby causing carbonates to mineralize within the pore spaces. At step 408, variation in differential pressure across the sample over time is measurements. Pressure buildup over time (or other characteristics of this time signal) can indicate a reduction in core permeability due to mineralization within the pore spaces, and the pressure differential signal can thus be analyzed to determine the effect of the treatment chemical on the degree, rate, and other aspects of the carbon mineralization.

Proceeding to step 412, a second aliquot of brine can be injected into the inner chamber and, at step 44, the final permeability of the sample after the treatment can be determined. Horizontal linear flow of an incompressible fluid is established through a core sample of length L and a cross-section of area A can be calculated using Darcy's equation:

$$Q = \frac{kA}{μ L} Δ P$$

In the above equation, Q is the flow rate of the fluid, A is the cross-sectional area of the fluid in contact with the formation, L is the length the fluid must travel through the formation, μ is fluid viscosity, k is permeability, and ΔP is the pressure differential along length L.

In some embodiments, after step 414, some or all of the carbon dioxide can be flowed from the chamber (mixed with other fluids from the chamber) and can be separated and re-injected as described with respect to steps 210, 212, and 214 of method 200 of FIG. 2.

FIG. 5 is a process flowchart of an example of a method 500 for determining a carbon sequestration characteristic of a geological material; more specifically, the effects of treating an organic-rich geological material with an oxidizer. In some embodiments, method 500 provides the specific sub-steps corresponding to step 208 of FIG. 2, with respect to such type of determination.

The method begins with step 502 with respect to a sample that has been placed into a sample holder chamber and a confining pressure applied (for example, in accordance with steps 202 and 204 of method 200 of FIG. 2). In the embodiment described with respect to FIG. 5, at step 502 organic material is added to the sample (for example, a sample manufactured from sand grains) after placement of the sample in the inner chamber by injecting asphaltene or another suitable hydrocarbon material into the inner chamber, thus resulting in an organic content that simulates the organic content of the geological material in question. For example, a sample can be manufactured from sand grains and commercial organic materials such as polymers. The polymers can be mixed in with the sand before packing or can be flowed into the sand pack by dispersing in solvent.

In other embodiments, the sample already contains the amount of organic matter in question (for example, a core sample of coal, source shale, or another organic-rich geological material). In some embodiments, the sand and organic materials (such as commercially-available polymers) are mixed together and then packed. At step 504, the sample is flushed with brine or another suitable fluid to eliminate excess organic material. In some embodiments, heat can be applied after step 504 or at another suitable time to simulate subsurface temperatures.

Proceeding to step 506, carbon dioxide is injected into the chamber. At step 508, the operator waits a suitable amount of time to allow for the injected carbon dioxide to saturate the sample, at the given pressure, temperature, and sample characteristics. At step 510, the pore pressure (the pressure of the fluid within the core sample) is measured after the time allotted to equalization has passed. At step 512, the volumetric storage capacity of the sample is determined based on the difference between these confining pressure measurements, for example, using the real gas law subjected to reservoir conditions.

At step 514, the carbon dioxide is flowed from the outlet of the chamber. In some embodiments, after step 514 and prior to step 516, some or all of the carbon dioxide can be flowed from the chamber (mixed with other fluids from the chamber) and can be separated and re-injected as described with respect to steps 210, 212, and 214 of method 200 of FIG. 2.

Proceeding to step 516, the oxidizer treatment material is injected. In some embodiments, the oxidizer treatment material can comprise oxidizers dissolved in water such as oxychlorine or oxybromine species (ClO⁻, ClO₂⁻, ClO₂, ClO₃⁻, ClO₄⁻, BrO⁻, BrO₂⁻, or BrO₃⁻. The oxidizer may be in the form of a salt such as a metal salt, preferably an alkali or alkaline earth metal salt. Examples of metal salts include sodium bromate NaBrO₃, sodium chlorite NaClO₂, potassium chlorate KClO₃, and the like. In some embodiments, the oxidizers are dissolved or dispersed in liquid carbon dioxide. Suitable oxidizers for dissolution or dispersion in CO₂ include ClO₂, Cl₂, Br₂, O₂, O₃, N₂O, NO, and NO₂. The ClO₂ can be prepared and captured using the chlorite/hydrochloric acid technique. In some embodiments, the oxidizers are dissolved in liquid carbon dioxide with a cosolvent. Suitable oxidizers for dissolution as cosolvent-modified CO₂ can include [Bu₄N]BrO₃ and [Bu₄N]ClO₃ and related oxidizers with hydrophobically modified cations and oxidizing anions. Suitable cosolvents to aid in their dissolution include alcohols such as ethanol, methanol, propanol and esters such as ethyl acetate and ethyl lactate.

Proceeding to step 518, carbon dioxide again is injected into the chamber. At step 520, the operator waits a suitable amount of time to allow for the injected carbon dioxide to saturate the sample, at the given pressure, temperature, and sample characteristics. At step 522, the pore pressure is measured after the time allotted to equalization has passed. At step 524, the volumetric storage capacity of the sample is determined based on the difference between these confining pressure measurements, for example, using the real gas law subjected to reservoir conditions. At step 526, the effectiveness of the oxidizer on carbon sequestration is determined based on the difference in the volumetric calculations done at steps 512 and 524.

In some embodiments, the oxidants utilized in the above methods are water-soluble salts that would be separated out and not recycled and reinjected with the carbon dioxide via return line 162. In some embodiments, the oxidant may be a gas such as ClO₂ or Br₂, in which case some or all of the oxidant would be recycled and reinjected along with the carbon dioxide.

A number of implementations of the disclosure have been described. Nevertheless, it will be understood that various modifications may be made without departing from the spirit and scope of the disclosure.

What is claimed is:

1. An apparatus for determining a carbon sequestration characteristic of a geological material, the apparatus comprising:
a sample holder comprising a fluid inlet, an inner chamber, and a fluid outlet, wherein the sample holder is configured to hold a sample representative of the geological material within the inner chamber and to permit a flow of fluid from the fluid inlet through the sample and thence out the fluid outlet;
a pressure pump configured to apply a confining pressure to the inner chamber;
a first pressure gauge and a second pressure gauge configured to measure a fluid pressure at the fluid inlet and a fluid pressure at the fluid outlet, respectively;
a carbon dioxide pump configured to inject carbon dioxide into the inner chamber via the fluid inlet;
a separator for separating a volume of carbon dioxide from fluid flowing from the fluid outlet; and
a return line configured to return at least a portion of the volume of carbon dioxide separated by the separator to the fluid inlet.

2. The apparatus of claim 1, further comprising a gas flow meter to measure a flow rate of the gas flowing from the fluid outlet to the separator.

3. The apparatus of claim 1, further comprising a treatment injection pump configured to inject a treatment chemical into the fluid inlet.

4. The apparatus of claim 3, wherein the treatment chemical comprises an oxidizer.

5. The apparatus of claim 4, wherein the oxidizer is water-soluble.

6. The apparatus of claim 1, wherein carbon dioxide injected by the carbon dioxide pump comprises a mixture comprising (a) carbon dioxide returned by the return line and (b) carbon dioxide from a carbon dioxide tank.

7. The apparatus of claim 6, wherein the carbon dioxide from the carbon dioxide tank has not been in contact with the sample before the injecting.

8. The apparatus of claim 1, further comprising a gas chromatograph-mass spectrometer configured to determine a composition of a fluid mixture flowing from the fluid outlet.

9. A method for evaluating a carbon sequestration characteristic of a geological material, the method comprising:
applying a confining pressure to an inner chamber of a sample holder, wherein a sample representative of the geological material is disposed in the inner chamber;
injecting carbon dioxide into the inner chamber via a fluid inlet of the sample holder, thereby flowing the carbon dioxide at least partially through the sample;
determining a carbon dioxide sequestration characteristic of the sample based, at least in part, on at least one of (a) a change in measured inner chamber pressure or (b) a change over time of a pressure differential across the sample;
flowing, out of the inner chamber via a fluid outlet of the sample holder, a fluid at least partially comprising the carbon dioxide flowed through the sample;
separating a volume of carbon dioxide from the fluid flowing from the fluid outlet; and
re-injecting at least a portion of the volume of carbon dioxide into the inner chamber.

10. The method of claim 9, wherein the carbon dioxide sequestration characteristic comprises a volumetric carbon dioxide storage capacity of the sample and wherein said volumetric storage capacity is based at least in part on a measured difference between measured inner chamber pressure before and after injection of the carbon dioxide.

11. The method of claim 9, wherein:
the geological formation of which the sample is representative comprises a saline-saturated formation;
the carbon dioxide sequestration characteristic comprises an increase in carbonate mineralization in the sample resulting from treatment of the sample by a treatment chemical; and
the method further comprises:
injecting a saline aqueous fluid into the inner chamber;
injecting the treatment chemical into the inner chamber;
measuring, over time, a pressure differential across the sample; and
determining, based at least in part on variations over time of the pressure differential, the increase in carbonate mineralization.

12. The method of claim 9, wherein:
the geological formation of which the sample is representative comprises an organic-rich formation;
the carbon dioxide sequestration characteristic comprises an increase carbon dioxide absorption resulting from treatment of the sample by an oxidizer;
the injecting carbon dioxide into the inner chamber comprises a first injection of carbon dioxide; and
the method further comprises:
determining a pre-treatment volumetric storage capacity based at least in part on a measured difference between measured inner chamber pressure before and after the first injection of the carbon dioxide;
flowing, from the inner chamber, at least a portion of the carbon dioxide from the first injection;
injecting an aliquot of the oxidizer into the inner chamber;
injecting carbon dioxide into the inner chamber as a second injection of carbon dioxide;
determining a post-treatment volumetric storage capacity based at least in part on a measured difference between measured inner chamber pressure before and after the second injection of the carbon dioxide; and
determining, based at least in part on a difference between the pre-treatment storage capacity and the post-treatment storage capacity, the increase in carbon dioxide absorption.

13. The method of claim 12, further comprising increasing the organic content of the sample by injecting a hydrocarbon fluid into the inner chamber prior to the first injection of carbon dioxide.

14. The method of claim 12, wherein the oxidizer is water-soluble.

15. The method of claim 9, a further comprising measuring, with a flow meter, a flow rate of the gas flowing through the sample chamber.

16. The method claim 9, wherein injecting carbon dioxide into the inner chamber comprises the reinjecting of the portion of the carbon dioxide separated by the separator.

17. The method of claim 16, wherein the portion of the carbon dioxide separated by the separator is injected as a mixture with carbon dioxide from a carbon dioxide tank.

18. The method of claim 17, wherein the carbon dioxide from the carbon dioxide tank has not been in contact with the sample before the injecting.

19. The method of claim 9, further comprising determining, with a gas chromatograph-mass spectrometer fluidically connected to the inner chamber, a composition of a fluid mixture flowing from the fluid outlet.

20. The method of claim 9, wherein the re-injecting the at least a portion of the volume of carbon dioxide into the inner chamber is via a return line fluidically connected to the fluid outlet.

21. The method of claim 9, wherein the return line is configured to flow the at least a portion of the volume of carbon dioxide to a pump configured to inject the carbon dioxide into the inner chamber via the fluid inlet.
